(19) United States
(12) Patent Application Publication
(10) Pub. No.: US 2014/0271689 A1
(43) Pub. Date: Sep. 18, 2014

(54) Therapeutic Cancer Vaccine Targeted to HAAH (Aspartyl-[Asparaginyl]-beta-Hydroxylase)

(71) Applicants: Biswajit Biswas, Germantown, MD (US); Carl R. Merril, Bethesda, MD (US); Hossein A. Ghanbari, Potomac, MD (US)

(72) Inventors: Biswajit Biswas, Germantown, MD (US); Carl R. Merril, Bethesda, MD (US); Hossein A. Ghanbari, Potomac, MD (US)

(73) Assignee: PANACEA PHARMACEUTICALS, GAITHERSBURG, MD (US)

(21) Appl. No.: 13/836,487

(22) Filed: Mar. 15, 2013

(51) Int. Cl. A61K 39/00; C12N 9/02

(52) U.S. Cl. CPC A61K 39/0011; C12N 9/0071
USPC 424/185.1; 435/235.1; 424/204.1; 536/23.2

(57) ABSTRACT

The present invention encompasses a cancer vaccine therapy targeting Aspartyl-[Asparaginyl]-β-hydroxylase (HAAH). The present invention contemplate bacteriophage expressing HAAH peptide fragments and methods for using said bacteriophage in methods of treating cancer.

### BACKGROUND OF THE INVENTION

Cancer is one of the most devastating diseases both in terms of human life opportunity loss and health care cost. It also presents unmet clinical needs. Currently available chemotherapies have limited efficacy and limited target patient population. Even the successful immunotherapies have shortcomings similar to chemotherapies. Moreover, essentially all cancer therapeutics have significant adverse side effects.

Aspartyl-(Asparaginyl)-β-hydroxylase (HAAH) is over expressed in various malignant neoplasms, including hepatocellular and lung carcinomas. HAAH is a tumor specific antigen, which is specifically expressed on the surface of certain malignant cells. HAAH is a hydroxylation enzyme that modifies factors such as Notch that contribute to cancer etiology by causing cell proliferation, motility, and invasiveness. Neutralizing the enzyme or reducing its expression leads to normal phenotype(s) in cancer cells. Anti-HAAH antibodies (as well as siRNA) have been shown to be cytostatic. An all-human sequence anti-HAAH (PAN-622) has shown to inhibit tumor growth by more than 90% in animal studies by passive immunotherapy. However, HAAH is well conserved and is also over expressed in placenta hence it is not sufficiently immunogenic in animals and it is certainly a self antigen in humans.

A vaccine therapy targeted to a pan-cancer-specific antigen such as HAAH that has proven relevance to cancer etiology is very desirable. Its economic impact will be enormous both in terms of job creation and increased productivity as well as in savings in health care and extending productive lives. The vaccine therapy of the present invention is novel both in terms of its target and the vaccine entity.

### SUMMARY OF THE INVENTION

The present invention encompasses a cancer vaccine therapy targeting human Aspartyl-[Asparaginyl]-β-hydroxylase (HAAH).

Certain embodiments of the present invention contemplate bacteriophage expressing HAAH peptide fragments, wherein the bacteriophage may be any one of Lambda, T4, T7, or M13/f1.

The present invention further contemplates methods of treating cancer comprising stimulating the immune system of a patient with bacteriophage expressing HAAH fragments.

The present invention also contemplates nano-particles comprising at least one amino acid sequence native to HAAH.

The present invention also encompasses methods for treating cancer comprising the step of providing an immune system stimulating amount of a Lambda phage to a patient, wherein the Lambda phage comprises amino acid sequences native to HAAH expressed on its surface.

The present invention also encompasses methods for treating cancer comprising the step of providing an immune system-stimulating amount of a nano-particle to a patient, wherein the nano-particle comprises amino acid sequences native to HAAH.

One embodiment of the present invention contemplates bacteriophage comprising at least one amino acid sequence native to HAAH, wherein the at least one amino acid sequence native to HAAH is selected from the group consisting of the amino acid sequence of Construct I, the amino acid sequence of Construct II and the amino acid sequence of Construct III.

The present invention also contemplates a Lambda phage expressing the amino acid sequence of Construct I, the amino acid sequence of Construct II or the amino acid sequence of Construct III on its surface.

Embodiments of the present invention also contemplate nucleic acid construct comprising at least one nucleotide sequence encoding an amino acid sequence native to HAAH and a nucleic acid sequence encoding bacteriophage lambda head decoration protein D (hereinafter “gpD”).

Another embodiment of the present invention includes nucleic acid constructs comprising nucleotide sequences encoding the amino acid sequence of Construct I, the amino acid sequence of Construct II or the amino acid sequence of Construct III.

### BRIEF DESCRIPTION OF THE FIGURES

FIG. 1 is a graph that demonstrates the efficacy of an antibody against HAAH in live cancer cells.

FIG. 2 shows the mechanism of immunization in accordance with the present invention.

FIG. 3 shows the immune response.

FIG. 4 Homologous recombination of donor plasmid pAN-A- with recipient phage vector. Only some of the lambda genes are shown. The unique Nhe I and Bssh II site in the lambda genome used for cloning is shown as is lacZa, a DNA cassette comprised of lacPO, RBS and the first 58 codons of lacZ. Generated recombinant phages are designated as HAAH construct I, II and III which contains an insert of HAAH fragment. Only diagram of construct I is shown here. The insert is fused with gpD head protein gene of lambda to produce gpD-HAAH construct I fusion on lambda capsid. The maps are not to scale.

FIG. 5. Example of a Western blot HAAH-vaccine screening for a cancer vaccine candidate.

FIG. 6 This scatter chart shows the result of HAAH test as a cancer biomarker on a group of 857 individuals composed of 211 individuals known not to have cancer and 646 patients who are diagnosed with cancer. The cancer group is composed of a mix of individuals with different types of cancer (Breast, Prostate, Lung, Colon) in various stages from one to four. Combining the 12 false positive and 34 false negative results, the test has less than 5.4% error even in such a large group of patients. Horizontal axis is the patient index.

FIG. 7 shows amino acid sequences in accordance with an embodiment of the present invention.

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

The present invention is based on the discovery that bacteriophage surface-expressed HAAH is highly immunogenic and could overcome tolerance of self antigen because of altered presentation and the adjuvant function of bacteriophage itself. The present invention provides a cancer vaccine therapy targeting HAAH using bacteriophage-expressed HAAH fragments.

It has been shown that passive immunotherapy using an all-human anti-HAAH is effective in cellular and animals models of cancer (in nude mice model, FIG. 1). The present invention demonstrates that bacteriophage delivery of HAAH fragments as vaccine can overcome the problem of self antigen tolerance by providing novel antigen presentation and inherent phage adjuvant properties.

In vitro activation of dendritic cells by tumor antigens, prior to administration to patient body shows promising results for cancer therapy. Unfortunately the process is cumbersome, expensive and time consuming for mass scale immune therapy against various cancers. Bacteriophage display is a simple way of achieving favorable presentation of peptides to the immune system. Previous findings revealed that recombinant bacteriophage can prime strong CD8+ T lymphocytes (CTLs) responses both in vitro and in vivo against epitopes displayed in multiple copies on their surface, activate T-helper cells and elicit the production of specific antibodies all normally without adjuvant.

As proposed herein, vaccination with lambda phage-displaying cancer specific antigen such as HAAH has a number of potential advantages. One of the advantages is display of multiple copies of peptides on the same lambda phage, and once the initial phage display has been made, subsequent production should be far easier and cheaper than the ongoing process of coupling peptides to carriers. There is also good evidence that due to particulate nature, phage-displayed peptides can access both the major histocompatibility complex (MHC) I and MHC II pathway, suggesting lambda phage display vaccines can stimulate both cellular and humoral arms of the immune system, although as extra cellular antigens, it is to be expected that the majority of the responses will be antibody (MHC class II) biased. It has been shown that particulate antigens, and phage in particular, can access the MHC I pathway through cross priming, and it is likely that it is this process which is responsible for stimulating a cellular response. This added cellular response mediated by CD8+ T cells helps to eliminate the cancer cells. Also, the role of Innate immunity in cancer is well established fact. Lambda phage can also act as nonspecific immune stimulators. It is likely that a combination of the foreign DNA (possibly due to the presence of CpG motifs) and the repeating peptide motif of the phage coat are responsible for the nonspecific immune stimulation. As a summary: whole lambda phage particles possess numerous intrinsic characteristics which make them ideal as vaccine delivery vehicles. For use as phage display vaccines, the particulate nature of phage means they should be far easier and cheaper to purify than soluble recombinant proteins since a simple centrifugation/ultra-filtration and column chromatography step should be sufficient to remove the majority of soluble contaminants. Additionally, the peptide antigen comes already covalently conjugated to an insoluble immunogenic carrier with natural adjuvant properties, without the need for complex chemical conjugation and downstream purification processes which must be repeated with each vaccine batch.

The present invention provides a prophylactic and therapeutic “phage vaccine” for both cancer prevention and treatment. In the present invention, fragmented HAAH peptides are successfully displayed on the surface of lambda head and large scale production and purification is carried out to perform animal experiments. The detail of these procedures is depicted below.

### A. Construction of Bacteriophage Lambda for Display of HAAH Peptides:

We designed a bacteriophage lambda system to display HAAH peptides fused at the C terminus of the head protein gpD of phage lambda. Molecular analysis of HAAH reveals a partial amino terminal homology of this protein with other two proteins called Junctin and Humbug. The role of these other two proteins in human physiology is not known completely. To avoid any complication such as activating immune system against these homologous proteins, we specifically eliminated these sequences from our phage display constructs. For proper display of HAAH peptides on lambda head, the rest of the HAAH sequence is segmented in three sections. They are designated as HAAH construct 1, HAAH construct 2 and HAAH construct 3 (see FIG. 7). Using HAAH specific oligo primers these segments are amplified from the HAAH gene which was previously cloned in our laboratory for expression in baculovirus system. The oligo sequence of each PCR primer is modified slightly to produce Nhe I and Bssh II restriction sites in each end of amplified HAAH segments. After restriction digestion, these segments are inserted separately at the NheI-BsshII site of the 3′ end of a DNA segment encoding gpD under the control of the lac promoter. The constructs are created in a plasmid vector (donor plasmid pAN-A), which also carries loxPwt and loxP511 sequences. Cre-expressing cells (E. coli) are transformed with these recombinant plasmids and subsequently infected with a recipient lambda phage that carries a stuffer DNA segment flanked by loxPwt and loxP511 sites. Recombination occurs in vivo at the lox sites and Ampr cointegrates are formed (FIG. 2), which are spontaneously lyse the E. coli and released in culture media. The cointegrates produce recombinant phages that display HAAH peptides fused at the C terminus of gpD. Approximately 200 copies of these peptides are displayed on a single phage head.

B. Selection of Lambda Cointegrates and Production of Recombinant Phages which Display HAAH Peptides:

Lambda cointegrates are selected on Luria Bartani (LB) ampicillin agar (100 ug/ml amp, 15% agar) plates. Briefly, spontaneously lysed E. coli culture is used to infect Cre-ve E. coli cells and spread on LB ampicillin agar plates. Plates are incubated at 32° C. for 48 hours to obtain Ampr colonies. These Ampr colonies are immune to super infection and carry the phages as plasmid cointegrates. The Ampr colonies containing the lambda cointegrate are grown separately at LB Ampicillin (100 ug/ml) at 37° C. for four hours. Lambda phages are spontaneously induced in these cultures and result in complete lysis. This cell free supernatant is used to infect E. coli cells and plated on solid LB agar (15%) plate to obtain phage plaques. The resulting phage plaques are harvested from the plate and single plaques are purified three times on E. coli by the standard procedures described by Sambrook et al.

### C. Conformation of Lambda Cointegrates Containing HAAH Fragments:

All bacterial colonies, containing lambda cointegrates, which are used for HAAH phage vaccine production are verified by PCR. In this process the presence of each cloned inserts in bacterial colonies are confirmed by PCR amplification of HAAH specific insert DNA by XbaI-5/(TTGGTTAGCAAGTTAATACC) and XbaI-3/(TAGATTTGAATGACTTCCCC) primer set. These two specific primers flank the unique Xba I site of lambda genome and used for PCR the complete insert presence in between Lox recombination sites of lambda DNA.

### D. Growth and Purification of Recombinant Phages Displaying HAAH Peptides:

Growth of the plaque purified phages is performed in two steps. The steps are designated as plate lysate method and large scale liquid lysate method. The detail of these procedures are described in Sambrook et al. The lysed culture is chilled at room temperature for further purification by liquid column chromatography technique.

### E. Large Scale Purification of Recombinant Lambda-Constructs Using Column Chromatography Technique:

CIM® monolithic columns are an ideal chromatographic support for purifying large biomolecules and nanoparticles, bacterial viruses and plasmid DNA. The pore size of these monolithic columns are adjusted to accommodate even the largest molecules and optimized for very high binding capacities at the highest flow rates. We adopted these monolithic columns for large scale purification of lambda phages displaying HAAH-peptides. In order to obtain infective virus during purification process we investigated chemical conditions that provided the maximal yield of phage and which also preserved high infectivity. This information is necessary to adjust chromatographic methods accordingly to avoid undesired phage deactivation during processing.

HPLC equipment: All experiment is preformed on a gradient AKTA purifier FPLC chromatography system (GE Healthcare) equipped with Unicorn 5.1 chromatography software, P-900 FPLC pumps, UPC-900 variable wavelength detector, and FRAC-920 fraction collector. CIM ion exchange chromatography is monitored for UV at 280 nm as well as for conductivity and the gradient profile, associated with marks for point of injection and fraction number. Stationary phase: A strong anion exchange (quaternary amine-QA) methacrylate-based CIM disk monolithic column (BIA Separations, Ljubljana, Slovenia) is used for this purification procedure. Mobile phase: 125 mM NaH₂PO₄, pH 7.0 (loading buffer) and 125 mM NaH₂PO₄, 1.5 M NaCl, pH 7.0 (elution buffer) of different pH values is used. All buffers is filtered through 0.22 micron pore size filters before use. These strong anion exchange (quaternary amine-QA) methacrylate-based CIM disk monolithic columns is periodically sanitized after processing, by a 2 hour procedure using 1 M NaOH. Processing of phage lysate for QA column analysis: Phage lysates (10 mL) are centrifuged at 12000×g for 10 minutes at 4° C. and the phage containing supernatant is filtered through a 0.22 micron filter prior to loading the phage on the column for chromatography. Collected fractions of 1 mL are analyzed via plaque assay to determine presence of infective phage. Plaque assay data is analyzed to optimize specific conditions for column chromatography purification of display phages. When larger amounts of highly concentrated phage will be required, the linear gradient will be changed into a stepwise gradient where narrower peaks will be achieved and fraction collection will be easier. Based on data from the linear gradient, we will introduce conditions for the stepwise gradient for large scale purification of display phages.

### F. Immunoblot and Western Blot Analysis of Recombinant Lambda-Constructs:

To verify the expression of fusion-peptides on lambda head, immunoblot and Western blot analysis are carried out.

For immunoblot assay each phage constructs are separately plated on LB agar plate to obtain 100 to 150 plaques in each plate. The plates are incubated at 37° C. for 18 hours, until the plaques are about one mm in size. Next, a 137 mm colony/plaque screen membrane (NEN® Research products, Boston, Mass.) is soaked in distilled water and blotted dry on a filter paper. This membrane is carefully placed on the top agar and incubation was continued at 37° C. for another 15 minutes. The membrane is peeled from the agar, and washed three times with Tris saline to remove the debris and bacteria. The plates are then stored at 4° C. and the washed NEN membranes are blocked with 2% casein solution for 1 hour. After blocking, the membranes are incubated in a casein solution containing 1.25 ug/ml of diluted FB 50 monoclonal antibody. This FB50 HAAH specific monoclonal antibody was previously generated in our laboratory for diagnostic application of prostate cancer. After incubation at room temperature for two hours the membranes are washed twice in Tris saline with 0.05% Triton X-100, and once in Tris saline for 15 minutes each. The monoclonal treated membranes are incubated with 2.0 μg/ml of alkaline phosphatase labeled rabbit antimouse IgG (Kirkegaard and Perry) for one hour at room temperature. The membranes are consecutively washed three times in the same way described earlier in this procedure, followed by a final wash with 0.9% NaCl. Finally the membranes are treated with Fast Red and naphthol substrate solution for about 10 minutes and the reaction was stopped by washing the membrane in distilled water. The pink immunoreactive spots corresponds the recombinants expressing HAAH specific peptides on lambda head. For Western blots, purified lambda phage particles were electrophoresed under reducing conditions on 0.1% (w/v) SDS/10% polyacrylamide gel followed by electroblotting onto PVDF membrane (Immobilon, Millipore, Bedford, Mass.). Fusion proteins are detected either 2.5 ug/ml diluted rabbit polyclonal sera raised against recombinant expressed lambda GpD or HAAH specific E6 mouse monoclonal antibody (final concentration 1.25 ug/ml). The rabbit antisera treated membranes are incubated with 2.0 μg/ml of alkaline phosphatase labeled goat anti-rabbit IgG and mouse monoclonal treated membranes are incubated with 2.0 μg/ml of alkaline phosphatase labeled rabbit antimouse IgG for one hour at room temperature. The membranes are consecutively washed three times in the same way described earlier in plaque lift assay. Finally the membranes are treated with Fast Red and naphthol substrate solution for about 10 minutes and the reaction is stopped by washing the membrane in distilled water. immunoreactive lines correspond to the gpD specific recombinant proteins.

### Animal Experiments to Evaluate Antigenic Nature of HAAH Phage Vaccine:

### A. Study of Antigenicity of HAAH-Phage Vaccine on Female BALB/c Mice.

The purpose of this experiment is to determine the efficacy of HAAH-phage vaccine to elicit antibody response in BALB/c female mice. Previously three separate HAAH-lambda phage constructs were prepared where fragmented HAAH antigens are displayed on surface of lambda phage head as fusion of lambda capsid protein gpD. Such three constructs were designated as HAAH construct 1, HAAH construct 2, and HAAH construct 3. Four separate groups of mice (Group A, Group B, Group C, 5 mice in each group and Group D, 40 mice) will be injected subcutaneously (s/c) with various HAAH phage constructs as described in chart below (Chart 1). Briefly, group A mice will receive 5×10⁸ pfu of HAAH construct 1 phage particles suspended in 500 μl of sterile PBS. Similarly group B and group C mice will receive same quantity of HAAH construct 2 and HAAH construct 3 phage particles respectively. Group D mice will receive equi-molar mixture of all 3 phage constructs. A fifth group of mice (group E, 40 mice) will receive recombinant HAAH antigen (50 μg/mice) suspended in sterile PBS. As a control (group F, 40 mice) will be injected with wild type phage pAN-A-λ. After primary inoculation, mice will receive 1st and 2nd booster (dose will be the same as primary inoculation) of corresponding antigens at 2 weeks interval. All animals will be bled prior primary inoculation. Serum samples will be collected before every booster to monitor progression of immune response against HAAH antigens. After 21 days animal will be euthanized for final bleeding through cardiac puncture. Finally animals will be sacrificed by spinal dislocations. Sera from group D, group E and group F animals will be saved at −70° C. freezer for second animal experiment. During experiment, all animals will be monitored for their health conditions. The immune response against various HAAH-phage vaccines will be monitored by western immunoblot and ELISA.

| | Groups | | | | | | |
|---|---|---|---|---|---|---|---|
| Days | A | B | C | D | E | F | Scoring |
| 0 | HAAH | HAAH | HAAH | Mixture of | Recombinant | pAN-A-λ | * |
| | construct 1 | construct 2 | construct 3 | 3 HAAH | HAAH | 5 × 10⁸ pfu | |
| | 5 × 10⁸ pfu | 5 × 10⁸ pfu | 5 × 10⁸ pfu | constructs | 50 μg | | |
| | | | | 5 × 10⁸ pfu | | | |
| 7 | HAAH | HAAH | HAAH | Mixture of | Recombinant | pAN-A-λ | |
| | construct 1 | construct 2 | construct 3 | 3 HAAH | HAAH | 5 × 10⁸ pfu | |
| | 5 × 10⁸ pfu | 5 × 10⁸ pfu | 5 × 10⁸ pfu | constructs | 50 μg | | |
| | | | | 5 × 10⁸ pfu | | | |
| 14 | HAAH | HAAH | HAAH | Mixture of | Recombinant | pAN-A-λ | |
| | construct 1 | construct 2 | construct 3 | 3 HAAH | HAAH | 5 × 10⁸ pfu | |
| | 5 × 10⁸ pfu | 5 × 10⁸ pfu | 5 × 10⁸ pfu | constructs | 50 μg | | |
| | | | | 5 × 10⁸ pfu | | | |
| 21 | Final Bleed | Final Bleed | Final Bleed | Final Bleed | Final Bleed | Final Bleed | * For 21 |
| | | | | | | | days |

Scoring: 0-normal, 1-lethargy and ruffled fur, 2-lethargy, ruffled fur and hunchback, 3-lethargy, ruffled fur, hunchback, and partially closed eyes, 4-moribund, 5-dead.

### B. Evaluation of Humoral Immunity Response Against HAAH Phage Constructs:

Previously in xenograft models of human primary liver cancer, the initial target disease, treatment with anti-HAAH antibodies reduced cancer tumor size in all animals, and in 75% of cases after four weeks of treatment tumors were kept to a non-detectable size. In a model of tumor metastasis using human colon cancer cells spreading to the liver, treatment with anti-HAAH antibodies greatly reduced the number and size of metastases. These results are highly significant and clearly indicate the utility of anti-HAAH in the treatment of human cancer. It is noteworthy that in both these instances animals were treated with antibody alone, not in conjunction with any other treatment. In this experiment, 4 groups of nude mice (Group A, Group B and Group C, and group D, 5 mice in each group) will be injected subcutaneously with a primary human liver cancer in their left flank. After 72 hours Group A, Group B and Group C nude mice will be treated by intraperitonial (i/p) route with 300 ul of sera previously collected from Group D, Group E and Group F mice of 1st animal experiment respectively. As a control Group D nude mice will be receive 300 ul of PBS. The treatment will continue every 48 hours for an additional 4 weeks. After that, the animal will be monitored for another 2 weeks without any intervention. The progression of the tumor will be monitored in treated and control groups every 48 hours to evaluate the result. Finally animals will be sacrificed by spinal dislocations and their organ will be examined by a pathologist for metastasis.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of compositions and processes would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A bacteriophage comprising at least one amino acid sequence native to Aspartyl-[Asparaginyl]-β-hydroxylase.

2. The bacteriophage of claim 1, wherein the at least one amino acid sequence native to Aspartyl-[Asparaginyl]-β-hydroxylase is selected from the group consisting of the amino acid sequence of Construct I.

3. The bacteriophage of claim 1, wherein the bacteriophage comprises the amino acid sequence of Construct II.

4. The bacteriophage of claim 1, wherein the bacteriophage comprises the amino acid sequence of Construct III.

5. The bacteriophage of claim 1, wherein the bacteriophage is selected from the group consisting of Lambda, T4, T7, and M13/f1.

6. The bacteriophage of claim 5, wherein the bacteriophage is bacteriophage Lambda.

7. A method for treating cancer comprising the step of providing a patient with an immune system stimulating amount of the bacteriophage of claim 1.

8. A nucleic acid construct comprising at least one nucleotide sequence encoding an amino acid sequence native to Aspartyl-[Asparaginyl]-β-hydroxylase and a nucleotide sequence encoding gpD.

9. The nucleic acid construct of claim 8, wherein the at least one amino acid sequence native to Aspartyl-[Asparaginyl]-β-hydroxylase is the amino acid sequence of Construct I.

10. The nucleic acid construct of claim 8, wherein the at least one amino acid sequence native to Aspartyl-[Asparaginyl]-β-hydroxylase is the amino acid sequence of Construct II.

11. The nucleic acid construct of claim 8, wherein the at least one amino acid sequence native to Aspartyl-[Asparaginyl]-β-hydroxylase is the amino acid sequence of Construct III.

12. A recombinant Lambda phage comprising the nucleic acid construct of claim 8.

13. A composition comprising nano-particles, wherein the nano-particles further comprise at least one amino acid sequence native to Aspartyl-[Asparaginyl]-β-hydroxylase.

14. The composition of claim 13, wherein the at least one amino acid sequence native to Aspartyl-[Asparaginyl]-β-hydroxylase is the amino acid sequence of Construct I.

15. The composition of claim 13, wherein the nano-particle comprises the amino acid sequence of Construct II.

16. The composition of claim 13, wherein the nano-particle comprises the amino acid sequence of Construct III.

17. A method for treating cancer comprising the step of providing a patient with an immune system stimulating amount of the composition of claim 13.

18. A method for treating cancer comprising the step of contacting dendritic cells of a patient with an immune system stimulating amount of the composition of claim 13.

19. A method for treating cancer comprising the step of providing an immune system stimulating amount of Lambda phage to a patient, wherein the Lambda phage comprise amino acid sequences native to Aspartyl-[Asparaginyl]-β-hydroxylase expressed on their surface.

20. The method of claim 19, wherein the amino acid sequences native to Aspartyl-[Asparaginyl]-β-hydroxylase comprise the amino acid sequence of Construct I, the amino acid sequence of Construct II and the amino acid sequence of Construct III.


SEQUENCE LISTING (6 sequences)

SEQ ID NO: 1 - DNA, 20 bp, Artificial Sequence
  Description of Artificial Sequence Synthetic primer
ttggttagcaagttaatacc

SEQ ID NO: 2 - DNA, 20 bp, Artificial Sequence
  Description of Artificial Sequence Synthetic primer
tagatttgaatgacttcccc

SEQ ID NO: 3 - protein, 314 aa, Artificial Sequence
  Description of Artificial Sequence Synthetic polypeptide
DRAMAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPP

SEQ ID NO: 4 - protein, 176 aa, Artificial Sequence
  Description of Artificial Sequence Synthetic polypeptide
LDAAEKLRKRGKIEEAVNAFKELVRKYPQSPRARYGKAQCEDDLAEKRRSNEVLRGAIETYQEVASLPDVPADLLKLSLKRRSDRQQFLGHMRGSLLTLQRLVQLFPNDTSLKNDLGVGYLLIGDNDNAKKVYEEVLSVTPNDGFAKVHYGFILKAQNKIAESIPYLKEGIESGDP

SEQ ID NO: 5 - protein, 238 aa, Artificial Sequence
  Description of Artificial Sequence Synthetic polypeptide
GTDDGRFYFHLGDAMQRVGNKEAYKWYELGHKRGHFASVWQRSLYNVNGLKAQPWWTPKETGYTELVKSLERNWKLIRDEGLAVMDKAKGLFLPEDENLREKGDWSQFTLWQQGRRNENACKGAPKTCTLLEKFPETTGCRRGQIKYSIMHPGTHVWPHTGPTNCRLRMHLGLVIPKEGCKIRCANETRTWEEGKVLIFDDSFEHEVWQDASSFRLIFIVDVWHPELTPQQRRSLPAI

SEQ ID NO: 6 - protein, 9 aa, Artificial Sequence
  Description of Artificial Sequence Synthetic peptide
HEFMQAWET